(19) United States
(12) Patent Application Publication
(10) Pub. No.: US 2002/0037258 A1
(43) Pub. Date: Mar. 28, 2002

(54) DENTAL COMPOSITION FOR THE MINERAL OCCLUSION OF DENTINAL TUBULES IN SENSITIVE TEETH

(76) Inventors: GREGORY P. DODD, HACKENSACK, NJ (US); ALAN A. HALECKY, WEST ORANGE, NJ (US); KENNETH J. MARKOWITZ, FANWOOD, NJ (US)

Correspondence Address: HANH T PHAM, BLOCK DRUG COMPANY INC, LAW DEPARTMENT PATENTS, 257 CORNELISON AVENUE, JERSEY CITY, NJ, 073023198

(21) Appl. No.: 09/419,514

(22) Filed: Oct. 18, 1999

Related U.S. Application Data
(60) Provisional application No. 60/147,438, filed on Aug. 5, 1999.

(51) Int. Cl. A61K 7/16; C09K 3/00; A61C 5/00

(52) U.S. Cl. 424/49; 424/401; 424/52; 106/35; 433/228.1

(57) ABSTRACT

A dental product composition for the treatment of sensitive teeth contains an amorphous bioactive particulate material consisting essentially of calcium, silicon and oxygen in an amount sufficient to alleviate tooth sensitivity by causing the formation of calcium containing mineral within the dentinal tubules of sensitive teeth.

This patent application claims the filing date of provisional patent application Serial No. 60/147,438 filed on Aug. 5, 1999.

### FIELD OF THE INVENTION

The present invention relates generally to compositions and methods for their use in the alleviation of dental pain associated with sensitive teeth.

### BACKGROUND OF THE INVENTION

Dentin is a hard but resilient calcareous tissue that mechanically supports the dental enamel. The dentin encloses and protects the pulp. The human tooth is comprised of a layer of enamel which is composed mainly of rods or prisms that are held together by an interprismatic cementing substance. The enamel covers the dentin which surrounds the pulp cavity and root canal. The root surface is covered by cementum The pulp cavity and root canal are filled with connective tissue which contains fibroblasts, histocytes, odontoblasts, blood vessels and nerves. The dentin also has a large number of continuous dentinal tubules which connect the pulp cavity with the dentinoenamel/cementum junction. These fine dentinal tubules radiate out from the pulp. In a sound tooth, the distal end of each tubule is generally covered by enamel or cementum, or by posteruptive deposits such as salivary mineral, or by a transient smear layer from mechanical burnishing. Posteruptive deposits may appear and disappear over the course of time as a consequence of various processes such as mineralization, demineralization, erosion, burnishing and abrasion.

The problem of tooth sensitivity arises when the enamel and cementum are worn away from the tooth by either improper oral hygiene practices or as a result of periodontal disease and/or its treatment. The dentinal tubules are fluid-filled exposing the central core of the tooth to the external environment. Hence, when subjected to air current, tactile or thermal stimulation, the pulpal nerves are irritated, leading to the perception of pain. It is well established in the scientific literature that the aforementioned stimuli evoke pain by inducing fluid movement within the dentinal tubules. In recent years, due to increased human longevity, an increasing number of people suffer from gingival degeneration and periodontitis. This trend magnifies the clinical significance of sensitive dentin.

The hydrodynamic theory is currently the predominant explanation of sensitive dentin. According to this theory, the stimuli conveyed through dentinal tubules irritates nerves distributed in the dental pulp causing pain in these areas. Suppressing sensitive dentin and ameliorating or alleviating the pain might be achieved by sealing the dentinal tubules, thus blocking the conductor of the sensation by physical or chemical means.

It is the fluid movement which in turn leads to activation of sensory nerve endings near the dentin/pulp boarder. The therapeutic approaches to treating dentin sensitivity involve either the reduction in the excitability of the intradental nerves by agents such as potassium ions or the reduction in the hydraulic conductance of the dentinal tubules by agents such as oxalate compounds as disclosed in U.S. Pat. No. 4,057,621. U.S. Pat. No. 5,037,639 discloses a method for the treatment of the pain associated with sensitive teeth through the application of compositions comprising calcium phosphate compounds that are formed via precipitation in situ. U.S. Pat. No. 5,027,031 discloses a dentifrice wherein the composition is rich in the salts of high molecular weight polyelectrolyte compounds. U.S. Pat. No. 5,250,288 teaches applying an effective amount of a charged polymeric particle to the surface of the tooth so that the particles enter and occlude the dentinal tubules. U.S. Pat. No. 5,244,651 discloses a composition with a colloid produced by mixing the salt of a polyvalent metal and a polyphosphate and/or a water-soluble salt thereof.

U.S. Pat. No. 5,330,746 discloses a dental varnish used to prevent bacterial plaque formation, periodontal disease and tooth sensitivity comprising an acrylic polymer, a hydrophilic polymer or a combination of the two with a strontium salt incorporated therein for long term sustained release. Other possible compounds for alleviating the pain associated with sensitive teeth include potassium, sodium and lithium nitrate (U.S. Pat. No. 3,863,006), potassium chloride and potassium bicarbonate (U.S. Pat. Nos. 4,631,185 and 4,751,072). U.S. Pat. No. 5,133,957 discloses a composition consisting of two co-polymerizable monomers which are polymerized in situ thereby occluding the dentin tubules. U.S. Pat. No. 5,374,417 discloses a potassium salt of a synthetic anionic polymer to close the dentinal tubules thus preventing the subsequent penetration of external stimuli to the dental pulp.

All of the above referenced materials use biologically inactive organic or inorganic components that will not sufficiently block or occlude the dentinal tubules so as to completely “desensitize” sensitive teeth. Normal habits including the eating of acidic foods and oral hygiene maintenance will remove these materials.

Applying a different approach to provide for at least partial occlusion of dentinal tubules, U.S. Pat. No. 5,735,942 (“'942 patent”) discloses a bioactive melt-derived glass composition for the treatment of tooth hypersensitivity. The '942 patent discloses the following composition by weight percentage: 40-60 SiO₂, 10-30 CaO, 10-35 Na₂O, 2-8 P₂O₅, 0-25 CaF₂ and 0-10 B₂O₃, with particle size of less than about 10 μm with some particles about 2 μm or less and some larger than 2 μm. The '942 patent notes that 60% SiO₂ is the maximum silica limit for bioactive melt-derived glass.

In a melt-derived process, there are three reactions which lead to the development of a porous hydrated silica-gel layer: ion exchange, silica dissolution, and condensation of silanols to form siloxane-bonded hydrated silica chains or rings. As the SiO₂ content increases in melt-derived glasses, the rates of these reactions decrease, reducing the availability of Ca⁺² ions in solution and the ability to develop the silica-gel layer on the surface. The result is the reduction and eventual elimination of the bioactivity of the melt-derived glasses as the SiO2 content approaches 60%. The process imposes narrow effective compositional zones which hinder the formulator's ability to modify and tailor the material for a specific application.

Bioactive glasses prepared by the melt-derived process and as taught in the '942 patent are processed in a platinum crucible at high temperatures, typically about 1350° C. or so. Therefore, melt-derived glasses are costly and difficult to transfer to production facilities. The high-temperature processing plus the multiple handling steps render bioactive glass prepared by the melt-derived process rather expensive, effectively limiting the practicality of using bioactive glass for oral care products.

Besides the high production cost, there are other disadvantages inherent with the '942 patent. First, it is difficult to maintain the very high purity required for optimal bioactivity due to the melt-derived process itself and the low silica and high alkali content of traditional bioactive glass compositions. Second, the '942 patent requires processing steps which involve grinding, polishing, fritting, sieving, etc., all of which expose the bioactive powder to potential contaminants that may negatively effect bioactivity. Third, there is a compositional limitation imposed on bioactive glasses and glass-ceramics made by conventional high-temperature processes due to the extremely high equilibrium liquidus temperature of SiO₂ of 1713° C., and the extremely high viscosity of silicate melts with high SiO₂ content.

U.S. Pat. No. 5,874,101 (the “'101 patent”) proposes a bioactive gel prepared by an improved process, a sol-gel process which includes a drying step for the treatment of tooth hypersensitivity. As with existing bioactive glasses and glass/ceramics and as disclosed in the the '942 patent, the bioactive glasses in the '101 patent are confined to a specific compositional zone in the Na₂O—CaO—P₂O₅—SiO₂ system defined mainly in silica content (Ogin, M., Ohuchi, F and Hench, L. L., Compositional dependence of the formation of calcium phosphate films on bioglass, J. Biomed. Mater. Res. 1980, 14, 55-64 and Ohtsuki, C., Kokubo, T., Takatsuka, K. and Yamamuro, T., Compositional dependence of bioactivity of glasses in the system CaO—SiO₂—P₂O₃: its in vitro evaluation, J. Ceram. Soc. Japn. 1991, 99, 1-6). The '101 patent discloses a composition in weight percent of 40-90 SiO₂, 4-45 CaO, 0-10 Na₂O, 2-16 P₂O₅, 0-25 CaF₂, 0-4 B₂O₃, 0-8 K₂O and 0-5 MgO.

There is still a need for a dental composition which is easy and inexpensive to prepare, and when lodged in the tubules of sensitive teeth, initiates the formation of calcium-containing mineral inside the dentinal tubules for the alleviation of the pain associated with sensitive teeth.

### SUMMARY OF THE INVENTION

This invention relates to an inorganic or organic/inorganic composite dental composition which consists essentially of CaO and SiO₂, when lodged in the tubules initiates the formation of calcium-containing mineral inside the dentinal tubules for the alleviation of the pain associated with sensitive teeth. The organic/inorganic composite material is preferably an amorphous binary oxide material of the system CaO—SiO₂ having been prepared by the addition of a soluble calcium source to a suitable silicon-donating precursor, such as TEOS or sodium silicate.

The dental composition of the invention may be produced and/or obtained by various methods, including but not limited to: a) a modified low-temperature sol-gel process producing inorganic composites; b) a modified low-temperature sol-gel process producing organically modified silicates; c) chemically or physically modifying naturally occurring analogs of the binary oxide material obtained from chemical supply companies, i.e.; calcium silicate derived from wollastonite; and d) the amorphous precipitation of inorganic materials.

### DETAILED DESCRIPTION OF THE INVENTION

The inventors have found a surprisingly low cost and convenient-to-manufacture bioactive materials that alleviates the pain associated with hypersensitive teeth. The inventors have also found a bioactive glass that is conducive to mineralization by varying the processing parameters and the wet chemistry of the materials.

Microscopic examination of exposed sensitive dentin surfaces show that clear differences exist between sensitive and non-sensitive regions of dentin. Sensitive dentin is permeated by open tubules. In contrast, non-sensitive tooth surfaces are characterized by tubules that are sealed off from the external environment by naturally occurring mineral deposits or coverings. We found that once bound to the dentin surface or lodged within the tubule, the amorphous material or morganic/organic materials of the present invention initiate nucleation and mineralization stimulated by the salivary environment, sealing the tubule from the external stimuli by depositing a layer of apatite or other calcium containing mineral similar to native tooth mineral in composition and crystallinity.

Bioactive Particulate Composition. Current bioactive materials, including bioactive glass, often rely on a complicated list of oxides, including P₂O₅, Na₂O, B2O₃, K₂O, MgO, Al₂O₃, TiO₂, Ta₂O₅ and fluoride salts, including CaF₂. We surprisingly found that nucleating crystal growth can be initiated by utilizing only a binary oxide system based exclusively on Si, O and Ca⁺². The bioactive materials prepared in accordance with the present invention, a CaO—SiO₂ containing inorganic/organic hybrid or inorganic composite derived from naturally occurring analogs, are capable of depositing a layer of apatite or other calcium-containing mineral, similar in composition and crystallinity to those mineral phases that predominate in normal dentin once the materials surface is placed in contact with blood, saliva or simulated body fluid.

We have also found that by retaining bioactivity while removing these species from the composition, we can lower the cost of the material, allow greater freedom in processing and eliminate possible organoleptic and toxicological problems. The simplicity of the chemical composition of the present invention, which not only aids in facilitating processing and lowering costs, but also allows flexibility to tailor the composition to the intended application. We found a material tailored to nucleating calcium-containing mineral for the treatment of a specific problem, tooth sensitivity, and removed excipients that may be a necessity for biological activity when used as an osteo-inductive material elsewhere in the body.

Unlike most currently-used bioactive materials that act in part by encouraging in-growth and tissue formation via osteoblasts and other hard tissue cells, the materials of the present invention surprisingly do not require cellular adjuvants. Additionally, the compositions in accordance with the present invention are able to induce the precipitation of apatite and calcium-containing mineral from simulated body fluids without the aid of bone morphogenic proteins, dentin phosphoproteins, odontoblasts or their host cells, or a defined collagen matrix. The ability to induce mineralization without the need for organic adjuvants optimizes this material for use in the oral environment.

In addition, many of these currently-used materials need highly specialized properties such as porosity and mechanical strength suited to their specific clinical use. For example, Li, Clark and Hench showed that there is a minimum rate of hydroxyapatite formation which is necessary to be an effective mineralizing agent and that this rate is a function of both composition and microstructure (Li et. al., ‘An Investigation of Bioactive Glass Powders by Sol-Gel Processing, J. of Applied Biomaterials, Vol. 2, 231-239 [1991]). The inventors have surprisingly found that the materials of the present invention do not need the porosity and mechanical strength of the prior art bioglasses to work in dental applications, so that when lodged in the tubules they initiate the formation of calcium-containing mineral inside the dentinal tubules.

The inventors have also surprisingly found that the bioactive materials of the present invention to be bioactive well beyond the compositional boundary for melt-derived glasses of the prior art with a less porous microstructure.

Additional Components. Carriers suitable for use with the composition are preferably hydroxylic materials such as water, polyols and mixtures thereof. Polyols, sometimes referred to as humectants, include glycerol, sorbitol, propylene glycol, xylitol, polypropylene glycol, polyethylene glycol, hydrogenated corn syrup and mixtures thereof Particularly preferred as the carrier is a liquid mixture of 3-30% water, 0-90% glycerol and 0-80% sorbitol. Generally the amount of the carrier will range from about 25 to 99.9% by weight, preferably from about 70-95% by weight.

When the compositions of this invention are in the form of a toothpaste or gel, there will typically be included a natural or synthetic thickening agent in an amount from about 0.1-10%, preferably about 0.5-5% by weight. Thickeners may include hydroxypropyl methylcellulose, hydroyethyl cellulose, sodium carboxymethylcellulose, xanthan gum, tragacanth gum, karaya gum, gum arabic, Irish moss, starch, alginates and carrageenans.

Surfactants are normally included in the oral compositions of this invention. These surfactants may be of the anionic, nonionic, cationic or amphoteric type. Most preferred are sodium lauryl sulfate, sodium dodecylbenzene sulfonate and sodium laurylsarcosinate. Surfactants are usually present in an amount from about 0.5-80% by weight.

For anti-caries protection, a source of fluoride ion will normally be present in the oral compositions. Fluoride sources include sodium fluoride, potassium fluoride, calcium fluoride, stannous monofluorophosphate, stannous fluoride and sodium monoflurophosphate. These sources should release from about 25-3500 ppm fluoride ion. The anti-caries agents will be present in an amount from about 0.05-3.0% by weight, preferably about 0.5-1.0%.

Flavors that are usually present in the oral compositions are those based on oils of IS spearmint and peppermint. Examples of other flavoring agents include menthol, clove, wintergreen, eucalyptus and aniseed. Flavors may range in concentration from about 0.1-5.0%.

Sweetening agents such as saccharin, sodium cyclamate, aspartame, sucrose and the like may be included at levels from about 0.1-5.0% by weight.

Other additives may be incorporated into the oral compositions including preservatives, silicones, other synthetic or natural polymers, anti-gingivitis actives, anti-tartar agents, whitening agents and other desirable products often found in a conventional toothpaste such as baking soda and peroxide.

It is also advantageous that these materials are compatible with conventional desensitizing agents such as potassium nitrate, potassium chloride and potassium bicarbonate, as well as novel desensitizing agents such as those described in U.S. Pat. No. 5,589,159.

Although it is not necessary, other oxides including P₂O₅, Na₂O, B₂O₃, K₂O, MgO, Al₂O₃, TiO₂, Ta₂O₅ and fluoride salts, including CaF₂, as commonly called for in the compositions in the prior art, can be added to the binary oxide system of the present invention.

Preparation. It is an advantage of the current invention that the materials are easy to fabricate eliminating the costly and time consuming processing procedures involved with the melt-derived and sol-gel derived bioactive materials in the prior art.

Compositions of the present invention can be prepared by several processes and with several chemical modifications designed to uniquely tailor the composition to a specific application because of advanced processing and by tailoring the wet chemistry to enhance the material's bioactivity. The processes of the present invention can be used to prepare the bioactive materials of the present invention, which are based exclusively on Si, O and Ca⁺², as well as bioactive materials which include a list of other oxides, including P₂O₅, Na₂O, B₂O₃, K₂O, MgO, Al₂O₃, TiO₂, Ta₂O₅ and fluoride salts.

1. Modified Sol-Gel Process Producing Inorganic Glasses. Sol-gel synthesis of glass is achieved by combining a metal alkoxide precursor with water and a catalyst with consequent polymerization of the metal alkoxide species and the production of a gel followed by the following steps: 1) heat sintering at temperatures often between 600-900 degrees centigrade, 2) calcination heat treatments, and often 3) supercritical drying to maintain the integrity of the nanostructured material.

Unlike glasses prepared by the melt-derived process, glasses prepared by the sol-gel process (“sol-gel-derived glasses”) maintain bioactivity for compositions up to pure silica gels. A particular characteristic of sol-gel processing is the production of microporous materials. As a result two advantageous consequences follow: 1) The high surface area associated with the porous calcium containing silica gel leads to a rapid increase in the concentration of Ca⁺² ions in the surrounding solution and 2) The texture produced by the sol-gel process results in a porous gel layer even with the reduced ion exchange and dissolution rates as the SiO₂ content increases. These two factors are responsible for the high rate of mineral formation and for the extension of the compositional bioactivity range in the sol-gel derived glasses. Studies have shown that sol-gel prepared silica, unlike melt-derived glass, has a highly hydrolyzed silica surface which facilitates and initiates apatite nucleation from metastable simulated body fluids.

The inventors have been able to surprisingly incorporate the favorable porosity and increased surface area associated with sol-gel derived materials in a “modified sol-gel process” that does not require the additional processing steps of heat sintering, calcination heat treatments, and supercritical drying to maintain the integrity of the nanostructured material, which steps are not easily adaptable to the production but necessary to achieve “a correctly formed sol-gel glass.”

In this modified sol-gel process of the invention to produce inorganic materials, an alkoxide precursor, a metal alkoxide of tetraethoxysilane (TEOS) and triethylphosphate (TEP), is hydrolyzed under acidic conditions and low temperatures to form a gel. Alkoxides of calcium, titanium, zirconium, magnesium, aluminum, iron and potassium can also be used. Other appropriate ingredients will also be apparent to those of ordinary skills in the art. The process can also be performed using alkaline conditions for the hydrolysis reaction. This has been found to control both the morphology and size of the powders produced.

2. Modified Sol-Gel Process Producing Organically Modified Silicates (“Ormosils”). In this Ormosils process, a hydrolysis reaction as described in the modified sol-gel process is first used to prepare organic/inorganic hybrid materials, followed by the formation of an inorganic network. During the build-up of this organic network, appropriately functionalized organic moieties also undergo a condensation reaction and are incorporated in the forming network.

In this second manifestation of the present invention, organic/inorganic hybrids are prepared from silanol terminated poly(dimethylsiloxane) [PDMS] and tetraethoxysilane [TEOS] precursors as described by Hu and MacKenzie (Journal of Material Science, 27:4415-4420 [1992]). The final particle size, surface modifications and control of reactivity can be controlled by varying processing parameters, specifically by the choice of catalyst and the pH of the system. It is advantageous that the flexibility of the materials can be controlled by changing the mixing ratios of the organic and inorganic components. If Ca(II) and other key species for bioactivity are introduced into the organic/inorganic hybrid material, these composites will exhibit both flexibility and bioactivity. The ability to control particle size, surface morphology, flexibility and bioactivity of the materials of the present invention enables the unique tailoring of these materials to their intended application.

3. Using/modifying Natural Minerals (Commercially Available Silicates). The bioactive agents of the present invention may be prepared by utilizing or modifying naturally occurring analogs of the inorganically produced material, i.e., calcium silicate obtained from wollastonite. Commercial soluble silicates have the general formula:

M₂OCₘSiO₂CnH₂O

where M is an alkali metal and m and n are the number of moles of SiO₂ and H2O, respectively, per mole of M₂O.

The composition of commercial silicates is typically described by the weight ratio of SiO₂ to M₂O. These soluble silicates have many uses, the largest and most rapidly growing of which arises from the ability to serve as a source of reactive primary silica species. The ability of this inorganic material to readily form the reactive silica species is the key to its ability to nucleate calcium-containing mineral. The rate of dissolution for soluble silicates depends on the glass ratio, solids concentration, production temperature, pressure, particle size and overall surface area. Dissolution typically occurs in a two-step mechanism that involves ion exchange and network breakdown.

Ion exchange: ≡Si—OCa+H2O←→≡Si—OH+Ca⁺²+OH⁻

Network breakdown: ≡Si—O—Si≡+OH←→≡Si—O⁻+HO—Si≡

As supplied, calcium silicate may not conform to the specifications or possess the functionality desired and certain modifications, i.e. surface alterations, particle size adjustment, and/or other necessary treatments may be implemented. When first received, the commercial material was moderately active in our evaluation However, after ball-milling the material to a mean size of 5 μm, the material provided enhanced bioactivity. Naturally occurring calcium silicate obtained from wollastonite has shown bioactivity in our in vitro evaluations.

These materials can be obtained commercially through major chemical supply companies including products from Alfa Aesar and Aldrich, including calcium silicate, meta (CaSiO₃, ortho (2CaOCSiO₂), etc.

4. Precipitation-Based Process Producing Inorganic Particles. The fourth manifestation of the present invention is our most preferred process. In this precipitation-based process, the sol-gel procedure is modified to a precipitation method to remove the difficult processing parameters inherent with a sol-gel (or the “modified sol-gel” method) simply by precipitating an amorphous oxide and not going through the ‘sol-gel’ stage. In this process, inorganic particles are produced as previously described in the sol-gel process, by the controlled hydrolysis of TEOS and resulting condensation incorporating reactive ions donated from a calcium source, either a calcium salt such as calcium nitrate or an alkoxide derivative such as calcium methoxide in a precipitation method The inorganic particles contain specific agents, ions, polymers or colloidal particles that render the materials of the present invention bioactive.

In a traditional sol-gel (or our “modified” sol-gel) procedure, silicate gels are often synthesized by hydrolyzing monomeric tetrafunctional alkoxide precursors employing a mineral acid (e.g., HCl) or base (e.g., NH₃) as a catalyst. At the functional group level, three reactions are generally used to describe the sol-gel process:

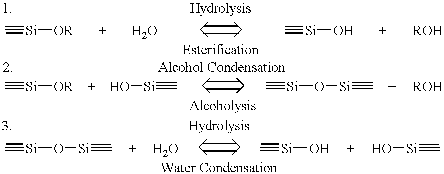

where R is an alkyl group, CₓH₂ₓ₊₁. The hydrolysis reaction replaces alkoxide groups (OR) with hydroxyl groups (OH). Subsequent condensation reactions involving the silanol groups produce siloxane bonds (Si—O—Si) plus the by-products alcohol (ROH) or water. Under most conditions, condensation commences before hydrolysis is complete.

Because water and alkoxysilane are immiscible, a mutual solvent such as alcohol is normally used as a homogenizing agent in a sol-gel process. However, gels can be prepared from silicon alkoxide-water mixtures without added solvent, since the alcohol produced as the by-product of the hydrolysis reaction is sufficient to homogenize the initially phase separated system. We have found that, if after the hydrolysis step, a reactive species, i.e.: calcium nitrate, is added to the mixing vessel, the silica will precipitate because of the surface charge destabilization within the sol. This precipitated material that when tested in our model, surprisingly achieves superior results as a dentinal tubule occluder and, as judged by scanning electron microscopy (“SEM”), a superior dentin mineralizing agent.

Not wishing to be bound by theory, it is postulated in our invention, Ca(II) and other key species for bioactivity may be introduced into the forming silica sol resulting in a particulate hybrid material. In general, the stabilization of colloids by electrostatic repulsion is successfully described by the DLVO theory (the Derjaguin, Landau, Verivery and Overbeak theory describing the stability suspensions) well-known to colloid chemists. Silica does not conform to the DLVO theory because it is apparently stabilized by a layer of adsorbed water that prevents coagulation even at the isoelectric point. The addition of cations to the aqueous silica sol may reduce the degree of hydration and destabilize the silica. Allen and Matijevic (J. Colloid and Interface Sci 31[3] 1969 287-296, 33[3] (1970) 420-429, 35 [1] (1971) 66-76) showed that adding a salt to the sol would produce ion exchange in the following manner:

≡Si—OH+M^{z+}→≡Si—OM^{(z-1)+}+H⁺

where M^{z+} is an unhydrolyzed cation of charge z.

Since the silanol groups are the adsorption sites for water, the removal of SiOH by ion exchange reduces the amount of hydration and lessens the stability of the colloid. The reaction precipitates a Ca(II)-containing silicate that can be easily removed from solution by filtration and resuspended in a vehicle suitable for application as a dental material. The remaining solutions may be reused facilitating both cost and production of the material.

In the precipitation-based process of the present invention, sodium silicate [CAS# 1344-09-8] may be substituted as the silicon source. The addition of an appropriate amount of a soluble calcium source, i.e. Ca(NO₃)₂, to a solution of sodium silicate precipitates a calcium-containing silicate whose composition is similar to previously described materials. The amount of Ca⁺⁺ to be added to the silicate solution was determined by both stoichiometric calculations and experimental design.

The precipitation-based process of the present invention has several advantages over other processes. This process produces no by-products that may contaminate the final product after incorporation into a standard dentrifice, i.e., ethanol or residual TEOS. In addition, the chemistry of the reaction in this process is such that particle size can more easily be controlled during the reaction, thus eliminating the need for further size reduction and milling steps which are not only costly but also may increase the chance for contamination of the final product. Further, the reaction requires only two ingredients, sodium silicate and the appropriate calcium source. Both of these chemicals are readily available from common chemical supply companies and are relatively inexpensive compared with the other processes.

In this most preferred process, the material, once precipitated and separated from the remaining solvent, does not require any special processing, including high heat or supercritical drying; nor does the material involve a calcination procedure or organic solvent extract. It is additionally preferred, although not critical, that in the process, the precipitated material is chemically controlled to produce amorphous material of sufficiently small particle size range as to not require additional milling, grinding or size reduction. Thus, one avoids the possibility of sample contamination and additional processing costs.

The preferred process of chemical reactions performed under ambient conditions produces a highly porous, calcium-containing silica similar to bioactive glass prepared by the traditional sol-gel process in biological activity. This inorganic material does not resemble the heat-sintered bioactive glass material currently available in other ways.

It is most preferred that the reaction produces a calcium-containing oxide material based solely on a two component system, CaO—SiO₂; and that the reaction occurs at room temperature in solvents familiar to the oral health care field as well as safe and well-known to production facilities personnel.

### EXAMPLES

The following examples are provided for illustrative purposes only, and changes or alterations may be made that are not disclosed therein.

In the examples, experiments were conducted using a modified in vitro model of dentin sensitivity described in U.S. Pat. No. 5,270,031. In this method, intact human third molars free of caries or restorations are sectioned perpendicular to the long axis of the tooth with a metallurgical saw into sections approximately 1 mm. thick. Sections containing dentin and free of enamel are retained for testing. These sections are then etched with an EDTA (ethylenediamine tetraacetic acid) solution to remove the smear layer. The disk is mounted in a split chamber device as reported in Journal of Dental Research 57:187 (1978). This special leak proof chamber is connected to a pressurized fluid reservoir containing a tissue culture fluid intended to mimic the osmolarity of human body fluid. By using a mixture of pressurized N₂ and CO₂ gas, the fluid can be maintained at physiological pH. The apparatus includes a glass capillary tube mounted on a ruler or other measuring instrument. An air bubble is injected into the glass capillary tube and by measuring the displacement of the bubble as a function of time the fluid flow through the dentin disk can be measured. It has been reported that fluid actually flows out of dentin tubules from the interior of a normal tooth

Following measurements of the baseline fluid flow in the dentin disk, the experimental mixture, dentifrice, gel or mouthwash is applied to the external disk surface in a manner that would mimic its use clinically. After a defined application period, the experimental material is rinsed off and post-application (lpacute) hydraulic conductance is measured. In this fashion various experimental materials both alone and as components of final products can be tested for the ability to obstruct fluid flow in the dental tubules. The percent flow reduction induced by application of the experimental material can then be calculated.

For all examples given, saliva incubation was necessary to achieve flow reductions. For the purpose of studying the mineralizing potential of novel agents, an extended time period is necessary as well as a modification of the standard dentin hydraulic conductance measurement. After initial post-application measurements are taken, the split chamber is removed from the apparatus and, a reservoir of sterile filtered human saliva is attached to the chamber allowing the fluid to bathe the treated occlusal dentin surface. The exterior of the chamber is wrapped in parafin to avoid contact with air and resulting fluid loss. The unit is then placed in an incubator kept at body temperature. After a sufficient time, the unit is removed, the saliva reservoir is discarded and the split chamber is re-attached to the hydraulic conductance apparatus. At this time, dentin fluid flow can be re-measured and the experimental material can be re-applied In the examples that follow, the flow reductions reported were obtained after three treatments and 40 hours of saliva incubation.

In the examples that follow as throughout this specification and claims, all temperatures are in degrees centigrade and all parts and percentages are by weight unless otherwise indicated.

### Example 1

### Modified sol-gel—ORMOSILS

The starting materials were reagent grade calcium nitrate, TEOS (tetraethoxysilane), PDMS (polydimethylsiloxane). Reagent grade 2-propanol and tetrahydrofuran were used as solvents and HCl was used as a catalyst. TEOS (10 g) and silanol terminated PDMS (5.9 g) were mixed with a mixture of 2-propanol (4.8 ml) and tetrahydrofuran (3.2 ml). This solution was denoted Solution A. An appropriate amount of calcium nitrate was dissolved in distilled water and HCl solution (35%). 2-propanol (8.0 ml) was added to the solution while stirring; this became Solution B. This recipe is based upon the synthesis of bioactive Ormosils outlined by K. Tsuru et al., Mat. Res. Soc. Symp. Proc. Vol. 435, p. 404, 1996.

Solutions A and B were mixed and subsequently refluxed while stirring at 80° C. for 30 minutes. After the reflux, the mixture was quenched to 25° C. with iced water, cast into containers and allowed to gel under ambient conditions. After the gelation, flat, irregularly shaped shards were milled to a known particle size. Milling can be performed by several methods including but not limited to ball-milling, air-impact milling, MICROS superfine milling, rotary cutter milling, hammer milling, and cage milling. This material, now of known particle size, was dispersed in glycerin and tested in our in vitro model as a slurry at various concentrations.

In the current embodiment of the invention, repeated applications of a 20% slurry showed good dentin fluid flow reducing ability as seen in Table 1.

TABLE 1
Percent Flow Reduction with Glycerin
Dispersions of an Organic/Inorganic Composite Material

| | MEAN POST- | | |
|---|---|---|---|
| | TREATMENT | STANDARD | SAMPLE SIZE |
| TREATMENT | (40 HRS) | DEVIATION | (N = ) |
| 20% Organic/Inor- | 61.5% | ±12.9 | 4 |
| ganic Composite | | | |
| Slurry in | | | |
| Glycerin | | | |

It should be noted that the cycle of repeated applications and filtered saliva incubations did not produce significant fluid flow reductions when non-bioactive materials were evaluated. In addition, the integrity of an untreated dentin surface and a placebo treated dentin surface, exposed to this cyclic method, was confirmed by SEM. No occlusion was observed when a finely ground pumice treated dentin surface was examined in this manner. Similarly, the morphology of untreated dentin surfaces that were exposed to the cyclic sterile saliva treatments alone, as in this mineralization model, did not significantly differ from freshly cut and prepared dentin specimens after SEM evaluation.

### Example 2

### Modifed sol-gel—ORMOSILS

The second embodiment of the present invention is prepared from the same precursors as in Example 1 and processed in a similar manner. However, in this example we significantly increased the total amount of calcium nitrate, the calcium ion source, in the material. Although this increase reduced the unusual mechanical strength and pliability of the original material, the resulting composition showed better dentin fluid flow reducin ability (Table 2) at various concentrations in glycerin dispersions.

TABLE 2
Percent Flow Reduction with Glycerin Dispersion
of an Organic/Inorganic Material with high calcium ion incorporation

| | MEAN POST- | | SAMPLE |
|---|---|---|---|
| | TREATMENT | STANDARD | SIZE |
| TREATMENT | (40 HRS) | DEVIATION | (N = ) |
| 5.0% Organic/Inorganic | 100% | 0.0 | 4 |
| Material in Glycerin | | | |
| 20% Organic/Inorganic | 100% | 0.0 | 4 |
| Material in Glycerin | | | |

This material resulted in a more rapid and complete occlusion than did the material described in Example 1.

### Example 3

### Modified sol-gel—Inorganic materials

A completely inorganic material is produced by a sol-gel process under ambient conditions. The material uses precursors similar to Example 2, but PDMS and tetrahydrofuran were omitted. The procedure is the same as in Examples 1 and 2. Although this material has poor mechanical strength, it is very effective as a tubule occluding agent. In addition, the absence of PDMS and tetrahydrofuran is favorable for the toxicological profile.

TABLE 3
Percent Flow Reduction with Glycerin
Dispersion of an Sol-Gel Derived Inorganic Composite

| | MEAN POST- | | SAMPLE |
|---|---|---|---|
| | TREATMENT | STANDARD | SIZE |
| TREATMENT | (40 HRS) | DEVIATION | (N = ) |
| 20.0% Sol-Gel Derived | 97.4% | ±3.7 | 8 |
| Inorganic Composite | | | |
| in Glycerin | | | |

### Example 4

### Modified sol-gel process—Base catalyzed reaction

A base-catalyzed sol-gel process is employed to control the shape and size of the resulting particulate suspensions. For these reactions, ammonia is used as a catalyst causing the formation of spherical particles. This procedure is based upon a 1968 Stöbber and Fink article, “Controlled Growth of Monodisperse Silica Spheres in the Micron Size Range” (Journal of Colloid and Interface Science, 26, 62-69, 1968). The resulting particulate composition proved to be a very effective mineralizing agent (Table 4). Scanning electron micrographs showed complete coverage of the dentinal tubules inferred to be due to mineralization of the tubule orifice in samples treated with the composite material. Untreated samples are marked by numerous patent tubules indicative of hypersensitive regions of the tooth. In addition, our ability to chemically control shape and morphology of the particulate material makes this example the easiest to formulate and process. In addition, elemental analysis of this material confirms large amounts of Ca⁺² and Si in a ratio conducive to bioactivity.

TABLE 4
Percent Flow Reduction with
Glycerin Dispersion of an Inorganic Composite

| | POST-MEAN | | SAMPLE |
|---|---|---|---|
| | TREATMENT | STANDARD | SIZE |
| TREATMENT | (40 HRS) | DEVIATION | (N = ) |
| 5.0% Inorganic Composite | 98.0% | ±3.4 | 3 |
| in Glycerin | | | |
| 10.0% Inorganic Composite | 97.7% | ±3.4 | 17 |
| in Glycerin | | | |

### Example 5

### Naturally Occuring Minerals

A calcium ortho-silicate [(2CaO—SiO₂), FW: 172.24, -325 mesh] derived from the mineral Wollastonite from Alfa Aesar was used. Published studies have indicated that pseudowollastonite, synthesized at 1500° C. for 2 hours from a stoichiometric mixture of calcium carbonate and silica, is bioactive in a simulated body fluid environment (De Aza PN. et al., Bioactivity of Pseudowollastonite in Human Saliva, Journal of Dentistry, 27 (1999), 107-113). As supplied from Alfa Aesar, the particle size was unsuitable for such an application and the material was ball-milled in glycerol to a mean particle size favorable to mineralization of a microporous substrate. As shown in Table 5, the resulting particulate composition proved to be an effective mineralizing agent.

TABLE 5
Percent Flow Reduction with Dispersions
of Calcium ortho-Silicate derived from Wollastonite

| | MEAN POST- | | |
|---|---|---|---|
| | TREATMENT | STANDARD | SAMPLE SIZE |
| TREATMENT | (40 HRS) | DEVIATION | (N = ) |
| 20% Naturally-derived | 74.3.% | ±30.0 | 4 |
| calcium ortho- | | | |
| silicate provided by | | | |
| Alfa Aesar | | | |

### Example 6

### Precipitation-Based Process Producing Inorganic Particles

A precipitate was prepared using commercially available calcium nitrate and sodium silicate solution. To 72.8 wt. % of a 40% sodium silicate solution, 27.2 wt. % of a 75% calcium nitrate tetrahydrate solution in deionized water was added, with the mixer running at high speed to provide maximum agitation to the sodium silicate solution Precipitation occurred immediately. The precipitate (Calcium Silicate, Inorganic Precipitate or “CSIP”) was dried at 60° C. for 40 hours. In example 6, the CSIP particle size was reduced by milling (e.g., using an air impact mill, simple ball mill, etc.). However, it is possible to alter the particle size by altering the pH or diluting the sodium silicate solution while maintaining the high calcium nitrate concentration of lowering the final particle size of the precipitate.

### Example 7

A toothpaste composition which further aids in the alleviation of pain when used on hypersensitive teeth was prepared using the following formulation. The desensitizing agent in this example can be any of the aforementioned Ormosil, inorganic composites, synthetically derived inorganic material, or sol-gel derived materials, or sodium silicate derived materials in a suitable composition. The weight percent given for each ingredient is based on a value of 100% for the total formulation.

| INGREDIENT | WEIGHT % |
|---|---|
| Water | 48.4 |
| Calcium Silicate (derived from a Sodium Silicate | 5.0 |
| Solution [1344-09-8] and Ca(NO₃)₂ [13477-34-4]) | |
| Sodium laurel sarcosinate | 0.4 |
| Sodium saccharin | 0.3 |
| Sodium fluoride | 0.2 |
| Hydroxyethylcellulose | 2.3 |
| Hydrated silica abrasive | 6.0 |
| Sodium lauryl sulfate | 1.2 |
| Glycerin | 23.0 |
| Hydrated silica thickener | 8.0 |
| Peppermint Oil | 0.2 |

### Example 8

A second toothpaste formulation was prepared comprising a desensitizing agent consisting of either an Ormosil, inorganic composite, synthetically derived inorganic material, or sol-gel derived materials. The weight percentage basis and the process of preparation are the same as that set forth in previous examples.

| INGREDIENT | WEIGHT % |
|---|---|
| Ormosil, containing high calcium | 3.0 |
| Sodium laurel sarcosinate | 0.6 |
| Sodium saccharin | 0.3 |
| Sodium fluoride | 0.3 |
| Hydroxyethylcellulose | 2.3 |
| Hydrated silica abrasive | 6.0 |
| Sodium lauryl sulfate | 1.2 |
| Glycerin | 71.1 |
| Hydrated silica thickener | 8.0 |
| Peppermint Oil | 0.2 |

### Example 9

A third toothpaste formulation was prepared as a two-phase system. This toothpaste may be in either one tube where each phase is kept separate by a septum dividing the package, it may be in a dual chamber pump that dispenses each phase simultaneously, or it may be packaged in separate tubes meant to be applied sequentially.

| | Phase A | |
|---|---|---|
| INGREDIENT | WEIGHT % | Phase B |
| Base-catalyzed inorganic composite | 3.0 | — |
| Sodium laurel sarcosinate | 0.6 | — |
| Hydroxyethylcellulose | 2.3 | 0.6 |
| Sodium lauryl sulfate | 1.2 | — |
| Glycerin | 82.8 | — |
| Hydrated silica thickener | 8.0 | 8.0 |
| Methyl Salicylate | 0.1 | — |
| Sodium fluoride | — | 0.3 |
| Sodium saccharin | — | 0.3 |
| Water | — | 84.6 |
| Peppermint Oil | — | 0.2 |
| Hydrated silica abrasive | — | 6.0 |

The active ingredient may also be formulated into a variety of dental rinse formulations designed to alleviate tooth sensitivity. An extremely fine particle size is needed to ensure homogeneous dispersion of either the Ormosil, inorganic composites, synthetically derived inorganic material, sol-gel derived materials or naturally-derived calcium silicate from mineral precursors. This can be accomplished by either chemical modification or mechanical milling the of material.

### Example 10

Formulations of a Medicinal-Type Mouthwash and Fluoride Oral Rinse are as follows in weight percent (adjusted to pH 6):

| INGREDIENT | Medicinal-Type | Fluoride Oral |
|---|---|---|
| Inorganic Precipitated Material | 1.0 | — |
| Ethyl Alcohol | 15.0 | — |
| Glycerin | 20.0 | 15.0 |
| Polyethylene glycol | 0.5 | — |
| Water | 61.5 | 73.5 |
| Caramel Color | to desired shade | — |
| Sodium Saccharin | 0.03 | 0.05 |
| Ormosil | — | 2.0 |
| Alcohol | — | 5.0 |
| Spearmint Oil | — | 0.25 |
| Poloxamer 338 | — | 1.0 |
| Sodium fluoride | — | 0.05 |
| Sodium benzoate | — | 0.1 |
| FD&C dyes | — | to desired color |

Although compositions in accordance with the present invention are effective with a single application, multiple applications will enhance effectiveness. Numerous vehicles are s present for the oral delivery of active agents of the present invention, including but not limited to, pastes, gels, rinses, powders, gums, dental floss, slurries and solutions and although each is not described it is within the scope of the present invention.

What is claimed is:

1. A dental product composition for the treatment of sensitive teeth comprising an amorphous bioactive particulate consisting essentially of calcium, silicon and oxygen, in an amount sufficient to alleviate tooth sensitivity by causing the formation of calcium containing mineral within the dentinal tubules of sensitive teeth.

2. The composition of claim 1 wherein said amorphous material is selected from the group consisting of:
a) a reaction product of an organic silicate source and a source of calcium;
b) a calcium containing hydrolysis product of tetraethylorthosilicate;
c) a calcium containing silica sol-gel;
d) a binary calcium oxide and silicate precipitated material;
e) a synthetic analog of a naturally occurring wollastonite-like calcium silicate; and
f) a precipitated reaction product of a soluble calcium source and a silicate solution.

3. The composition of claim 2 comprising a carrier and about 1-25% of said amorphous material.

4. The composition of claim 2 wherein the carrier comprises 0-30% water, 0-90% glycerol and 0-80% sorbitol.

5. The composition of claim 2 wherein said amorphous material is about 5-10% of the composition and the composition is a dentifrice.

6. The composition of claim 4 containing about 0.1-10% thickener, about 0.5-80% surfactant about 0.05-3% anti-caries agent, about 0.1-5% flavor and about 0.1-5% sweetening agent.

7. A method for the treatment of sensitive teeth comprising contacting said teeth with an amorphous bioactive particulate consisting essentially of calcium, silicon and oxygen, in an amount sufficient to alleviate tooth sensitivity by causing the formation of calcium containing mineral within the dentinal tubules of the sensitive teeth.

8. The method of claim 7 wherein said amorphous material is selected from the group consisting of:
f) a reaction product of an organic silicate source and a source of calcium;
g) a calcium containing hydrolysis product of tetraethylorthosilicate;
h) a calcium containing silica sol-gel;
i) a binary calcium oxide and silicate precipitated material;
j) a synthetic analog of a naturally occurring wollastonite-like calcium silicate; and
f) a precipitated reaction product of a soluble calcium source and a silicate solution.

9. The method of claim 8 comprising contacting said teeth with a composition comprising a carrier and about 1-25% of said amorphous material.

10. The method of claim 8 wherein said teeth are contacted with a dentifrice containing about 5-10% of said amorphous material.

11. A process for producing an amorphous bioactive particulate for the treatment of sensitive, said process consisting essentially of the following steps: adding a sufficient amount of a soluble calcium source to cause a precipitation of calcium-containing silicate particulates.

12. The process of claim 11, wherein said soluble calcium source is selected from the group consisting of calcium salts and alkoxide derivatives.

13. A process to produce of an amorphous bioactive particulate for the treatment of hypersensitive teeth, said process consisting essentially of combining a metal alkoxide precursor with water and a catalyst and polymerization of the metal alkoxide species for the production of said amorphous bioactive particulate.

14. The process of claim 13, wherein said metal alkoxide precursor is tetraethoxysilane.

15. The process of claim 13, wherein said metal alkoxide precursor is prepared from tetraethoxysilane and silanol terminated poly(dimethylsiloxane).
